(19) United States
(12) Patent Application Publication
(10) Pub. No.: US 2021/0324084 A1
(43) Pub. Date: Oct. 21, 2021

(54) METHOD FOR SUPPRESSION OF HEPAPITIS B VIRUS REPLICATION AND HEPAPITIS B VIRUS SURFACE ANTIGEN SECRETION

(71) Applicant: Ascendo Biotechnology, Inc., Taipei (TW)

(72) Inventors: Yen-Ta LU, Taipei (TW); Ping-Yen HUANG, Taipei (TW); Chia-Ming CHANG, Taipei (TW); I-Fang TSAI, Taipei (TW); Frank Wen-Chi LEE, Bedford, MA (US); Huei-Ling CHANG, Taipei (TW)

(73) Assignee: Ascendo Biotechnology, Inc., Taipei (TW)

(21) Appl. No.: 17/267,483

(22) PCT Filed: Aug. 9, 2019
(86) PCT No.: PCT/US2019/046064

§ 371 Date: Feb. 9, 2021

Related U.S. Application Data
(60) Provisional application No. 62/716,375, filed on Aug. 9, 2018.

(51) Int. Cl. C07K 16/28; A61P 31/14

(52) U.S. Cl. CPC C07K 16/2845; A61K 2039/505; A61P 31/14

(57) ABSTRACT

A pharmaceutical composition for use in treating hepatitis B virus (HBV) infection includes an effective amount of an antibody against CD11b or a binding fragment thereof. A method for treating hepatitis B virus infection includes administering to a subject in need thereof an antibody against CD11b. Anti-CD11b antibody binding to CD11b may trigger immunostimulatory responses, as evidenced by the following observations: increased surface expression of MHC II and CD86 in CD11b+ peripheral blood mononuclear cells (PBMCs); suppressed level of hepatitis B surface antigen (HBsAg) and HBV DNA in the blood; and accelerated clearance of HBV from liver.

### FIELD OF THE INVENTION

The present invention relates to the field of liver immunotherapy, particular to immune clearance of hepatitis B virus infection.

### BACKGROUND OF THE INVENTION

Hepatitis B virus (HBV) is a major human pathogen that causes acute and chronic hepatitis and hepatocellular carcinoma (HCC). Although an effective HBV vaccine is available, over 240 million people worldwide are estimated to be chronically infected by HBV. The untreated individuals serve as virus carriers and have a high risk of developing cirrhosis and HCC. The present treatment regimens for chronic hepatitis B, involving pegylated interferon and nucleos(t)ide analogues (lamivudine, adefovir, entecavir, and tenofovir etc.), can suppress HBV DNA replication. However, only about 3%-7% of patients treated with pegylated interferon and 1%-12% of patients treated with nucleos(t)ide analogues showed a sustained response. In addition, treatment with nucleos(t)ide analogues may induced drug-resistant HBV variants. Thus, other therapeutic strategies for the treatment of chronic HBV infection need to be explored.

The liver is the largest internal organ in the body, responsible for detoxification, metabolic activities, and nutrient storage. In additions, the liver is an immunological organ with unique properties, including predominant innate immunity, less adaptive immunity and induction of immune tolerance. Thus, the liver usually fails to exert effective immune responses to clear many important pathogens, such hepatitis B virus (HBV), hepatitis C virus (HCV), or malaria. These pathogens can evade immune surveillance and sustain persistent infections in the hepatic microenvironment. It is critical to reverse immune tolerance of liver for complete clearance of persistent infection.

CD11b is a type I transmembrane glycoprotein expressed on surface of hepatic immune cells, including Kupffer cells (liver-resident macrophages), dendritic cells (DCs), myeloid-derived suppressor cells (MDSC), nature killer cells (NK), and subsets of B and T cells. CD11b is also called integrin alpha M (ITGAM), which non-covalently binds with its (3-chain partner, CD18, to form the functional integrin heterodimer CD11b/CD18. CD11b/CD18 is also called macrophage-1 antigen (Mac-1) or complement receptor 3 (CR3), which mediates inflammation, by regulating cell adhesion, migration, chemotaxis, and phagocytosis.

Recent studies have shown that activated CD11b negatively regulates TLR signaling through ubiquitin-mediated degradation of MyD88 and TRIF (C. Han et al., Nat. Immunol., 2010, 11(8): 734-42). Activated CD11b also negatively regulates DC function to suppress T cells activation and negatively regulates B-cell receptor (BCR) signaling to maintain B cell tolerance.

### SUMMARY OF THE INVENTION

The present invention relates to methods for modulating immune response based on binding CD11b on the hepatic myeloid and lymphoid immune cell populations. Particularly, binding to CD11b with anti-CD11b antibody triggers immunostimulatory environment that has one or more of the following effects: increasing surface expression of MHC II and CD86 on CD11b+ peripheral blood mononuclear cells (PBMCs); suppressing the level of hepatitis B surface antigen (HBsAg) and HBV DNA in the blood; and accelerating clearance of HBV from liver.

One aspect of the invention relates to pharmaceutical compositions for use in treating hepatitis B virus infections. A pharmaceutical composition in accordance with one embodiment of the invention comprises an effective amount of an antibody against CD11b or a binding fragment thereof. An effective amount is that which will produce the desired effects. One skilled in the art would appreciate that the effective amount would depend on the patient's conditions, age, gender, etc. and the effective amount can be determined using routine skills without undue experimentation. A binding fragment from an antibody may include, but are not limited to, Fv, Fab, Fab′, Fab′-SH, F(ab′)2; diabodies; linear antibodies; single-chain antibody molecules (scFv); and multi-specific antibodies formed from antibody fragments.

In accordance with embodiment of the invention, an antibody against CD11b may be a polyclonal or monoclonal antibody. The antibody against CD11b may comprise a heavy-chain complementarity determining region 1 (HCDR1) consisting of the amino acid residues of NYWIN (SEQ ID NO:1) or GFSLTSNSIS (SEQ ID NO:2); a heavy chain CDR2 (HCDR2) consisting of the amino acid residues of NIYPSDTYINHNQKFKD (SEQ ID NO:3) or AIWSGGGTDYNSDLKS (SEQ ID NO:4); and a heavy chain CDR3 (HCDR3) consisting of the amino acid residues of SAYANYFDY (SEQ ID NO:5) or RGGYPYYFDY (SEQ ID NO:6); and a light chain CDR1 (LCDR1) consisting of the amino acid residues of RASQNIGTSIH (SEQ ID NO:7) or KSSQSLLYSENQENYLA (SEQ ID NO:8); a light chain CDR2 (LCDR2) consisting of the amino acid residues of YASESIS (SEQ ID NO:9) or WASTRQS (SEQ ID NO:10); and a light chain CDR3 (LCDR3) consisting of the amino acid residues QQSDSWPTLT (SEQ ID NO:11) or QQYYDTPLT (SEQ ID NO:12).

In accordance with some embodiments of the invention, the antibody against CD11b comprises: (a) a heavy chain variable region comprising the sequence of SEQ ID NO:13, and a light chain variable region comprising the sequence of SEQ ID NO:23; (b) a heavy chain variable region comprising the sequence of SEQ ID NO:14, and a light chain variable region comprising the sequence of SEQ ID NO:24; (c) a heavy chain variable region comprising the sequence of SEQ ID NO:15, and a light chain variable region comprising the sequence of SEQ ID NO:25; (d) a heavy chain variable region comprising the sequence of SEQ ID NO:16, and a light chain variable region comprising the sequence of SEQ ID NO:26; (e) a heavy chain variable region comprising the sequence of SEQ ID NO:17, and a light chain variable region comprising the sequence of SEQ ID NO:27; (f) a heavy chain variable region comprising the sequence of SEQ ID NO:18, and a light chain variable region comprising the sequence of SEQ ID NO:28; (g) a heavy chain variable region comprising the sequence of SEQ ID NO:19, and a light chain variable region comprising the sequence of SEQ ID NO:29; (h) a heavy chain variable region comprising the sequence of SEQ ID NO:20, and a light chain variable region comprising the sequence of SEQ ID NO:30; (i) a heavy chain variable region comprising the sequence of SEQ ID NO:21, and a light chain variable region comprising the sequence of SEQ ID NO:31; or (j) a heavy chain variable region comprising the sequence of SEQ ID NO:22, and a light chain variable region comprising the sequence of SEQ ID NO:32.

One aspect of the invention relates to methods for treating HBV infections. A method in accordance with one embodiment of the invention comprises administering to a subject in need thereof an effective amount of an antibody against CD11 b. Anti-CD11b antibody binding to CD11b triggers immunostimulatory responses, as evidenced by the following observations: increased surface expression of MHC II and CD86 in CD11b+ peripheral blood mononuclear cells (PBMCs); suppressed level of hepatitis B surface antigen (HBsAg) and HBV DNA in the blood; and accelerated clearance of HBV from liver

### BRIEF DESCRIPTION OF THE DRAWINGS

FIG. 1 shows a schematic diagram depicting a treatment protocol in accordance with one embodiment of this invention.

FIG. 2 shows surface expression of MHC II and CD86 on CD11b+ peripheral blood mononuclear cells (PBMCs) in hydrodynamic injection-based HBV carrier mice after antibody treatments.

FIG. 3 shows dynamic change of serum HBsAg in hydrodynamic injection-based HBV carrier mice after antibody treatments. Data are shown as mean±SEM (*p<0.05, Student's t test).

FIG. 4 shows dynamic change of serum HBV DNA in hydrodynamic injection-based HBV carrier mice after antibody treatments. Data are shown as mean±SEM (*p<0.05, **p<0.01, Student's t test).

FIG. 5 shows relationship among the level of serum HBV DNA, MHC II, and CD86 expressions on CD11b+ PBMCs in hydrodynamic injection-based HBV carrier mice after antibody treatments. Correlations were determined using the Pearson's correlation coefficient.

FIG. 6A shows the expression of CD11b on HepG2 cells. FIG. 6B shows the titer of HBsAg, and FIG. 6C shows the titer of apolipoprotein B mRNA editing enzyme, catalytic polypeptide-like (APOBEC-B) RNA expression of HBV-transfected HepG2 cells after anti-CD11b antibody treatment. Data are shown as mean±SEM.

FIG. 7 shows results of quantification of HBV DNA in liver. Total liver DNA was extracted and 1 μg of gDNA was measured by real time PCR with HBx specific primer. Each dot represents HBV DNA from 1 mouse liver. The detected limitation is 1000 copies/μg.

FIG. 8 shows light chain variable region sequences for 10 humanized anti-CD11b antibodies.

FIG. 9 shows heavy chain variable region sequences for 10 humanized anti-CD11b antibodies.

FIG. 10 shows the bindings of the 10 humanized anti-CD11b antibodies to CD11b expressed on K562 cells as analyzed with flow cytometry.

### DETAILED DESCRIPTION

Embodiments of the present invention relate to methods for treating or alleviating conditions of HBV infections. Methods of the invention are based on modulating immune responses by antibody, or a binding fragment thereof, bindings to CD11b on the hepatic myeloid and lymphoid immune cell populations. Inventors of the invention unexpected found that bindings to CD11b with anti-CD11b antibodies trigger immunostimulatory environment that has one or more of the following effects: increasing surface expression of MHC II and CD86 on CD11b+ peripheral blood mononuclear cells (PBMCs); suppressing the level of hepatitis B surface antigen (HBsAg) and HBV DNA in the blood; and accelerating clearance of HBV from liver.

Hepatitis B virus (HBV) is an enveloped virus with a covalently closed circular double-stranded DNA (cccDNA) genome. HBV infection causes acute and chronic inflammatory liver diseases. Long-term HBV infection can cause hepatic cirrhosis and hepatocellular carcinoma. The long-term chronic infection of HBV results from impaired HBV-specific immune responses, thereby the immune system fails to eliminate or cure the infected hepatocytes.

CD11b is a type I transmembrane glycoprotein expressed on surface of hepatic immune cells, including Kupffer cells (liver-resident macrophages), dendritic cells (DCs), myeloid-derived suppressor cells (MDSC), nature killer cells (NK), and subsets of B and T cells. CD11b is also called integrin alpha M (ITGAM), which non-covalently binds with its (3-chain partner, CD18, to form the functional integrin heterodimer CD11b/CD18. CD11b/CD18 is also called macrophage-1 antigen (Mac-1) or complement receptor 3 (CR3), which mediates inflammation, by regulating cell adhesion, migration, chemotaxis, and phagocytosis.

In systemic lupus erythematosus, a variant of integrin-αM (CD11b variant) is associated with autoreactive B cells that exhibit hyperproliferative response to B cell receptor (BCR) crosslinking. Using B cells transfected with the wild type or lupus-associated variant of CD11b, Ding et al. found that the mutation in the variant CD11b abrogates the regulatory effect of CD11b on BCR signaling, by disruption of CD22-CD11b direct binding. (C. Ding et al., Nat. Commun. 2013; 4:2813). They conclude that CD11b negatively regulates BCR signaling to maintain autoreactive B cell tolerance.

However, CD11b may play different roles in different systems or diseases. For example, CD11b deficiency enhances TLR-mediated responses in macrophages, rendering mice more susceptible to endotoxin shock and Escherichia coli-caused sepsis, suggesting CD11b negatively regulates TLR signaling through ubiquitin-mediated degradation of MyD88 and TRIF (C. Han et al., Nat. Immunol., 2010, 11(8): 734-42). It is not known whether integrin-aM (CD11b) plays any role in liver diseases, such as HBV infections.

Thus, inventors of the invention set out to investigate whether CD11b plays any role in HBV infections. We unexpectedly found that CD11b indeed plays a role in hepatic immune responses to chronic HBV infection. Briefly, inhibition of CD11b functions by binding anti-CD11b antibodies to CD11b resulted in immunostimulatory responses, as evidenced by increased surface expressions of MHC II and CD86 in CD11b+ peripheral blood mononuclear cells (PBMCs), suppressed levels of hepatitis B surface antigen (HBsAg) and HBV DNA in the blood, and accelerated clearance of HBV from liver.

Based on these unexpected findings, embodiments of the invention relate to methods for controlling or treating or alleviating conditions of HBV infections. Methods of the invention are based on antibody bindings to CD11b, particularly CD11b on hepatic myeloid cells and lymphoid immune cells. Embodiments of the invention will be illustrated with the following specific examples. One skilled in the art would appreciate that these examples are for illustration only and are not meant to limit the scope of the invention because other modifications and variations are possible without departing from the scope of the invention.

### Anti-CD11b Antibodies

Embodiments of the invention may use various anti-CD11b antibodies, which may be polyclonal or monoclonal and include commercially available antibodies. Several anti-CD11b antibodies are commercially available from various vendors. For example, CD11b monoclonal antibody (M1/70), CD11b monoclonal antibody (M1/70.15), and CD11b monoclonal antibody (ICRF44) are available from Thermo Fisher Scientifics (Waltham, Mass., USA) among others. Embodiments of the invention may use any of these commercially available anti-CD11b antibodies or a CD11b binding fragment thereof.

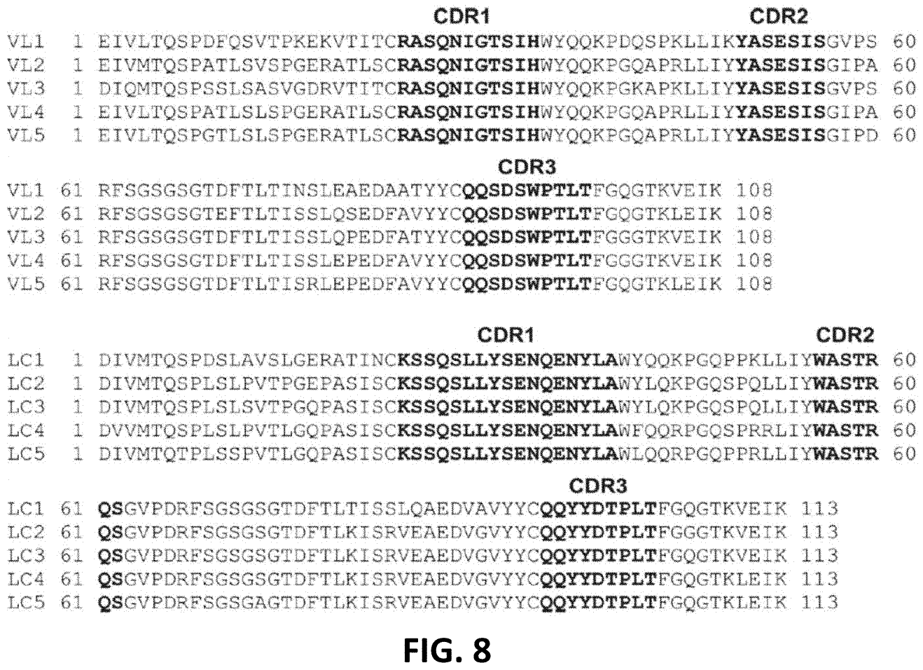

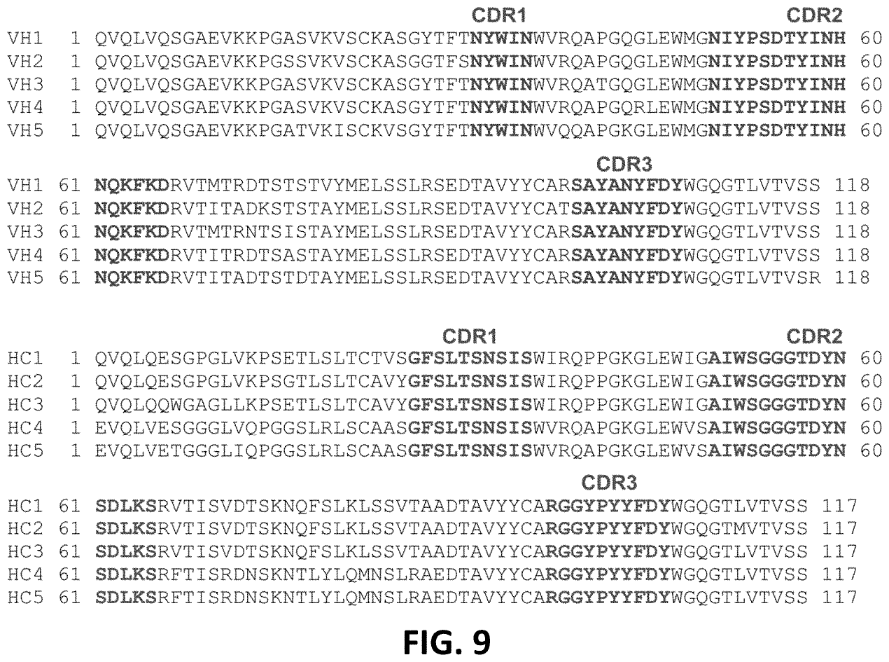

In addition, we have generated several monoclonal antibodies and humanized antibodies that bind specifically to CD11b. These antibodies were found to have similar biological activities. The production of monoclonal antibodies and humanization of antibodies use techniques known in the art (see US 2018/0362651A1, the disclosure of which is incorporated by reference). For humanization, the variable domain sequences of murine anti-human CD11b antibody were searched against a human antibody database. As an example, 10 sets of human framework sequences with high homologies to murine anti-human CD11b were chosen as human acceptors for both light and heavy chains. Meanwhile, N-glycosylation motifs were analyzed. Potential glycosylation sites in the candidate human variable regions should therefore be avoided. The humanized variable domains of 10 light chains were denoted as VL1, VL2, VL3, VL4, VL5, LC1, LC2, LC3, LC4, and LC5 (FIG. 8); while the humanized variable domains of 10 heavy chains were denoted as VH1, VH2, VH3, VH4, VH5, HC1, HC2, HC3, HC4, and HC5 (FIG. 9). These light chain and heavy chain peptide sequences provide humanized antibodies or antigen-binding portions that bind to human anti-CD11b with high affinity.

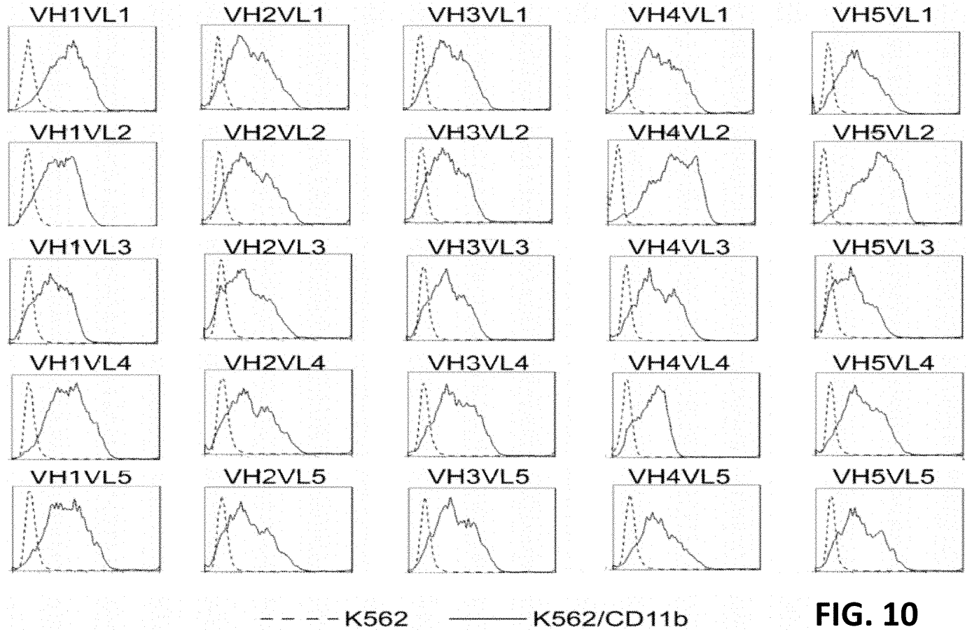

The specificities of humanized anti-CD11b antibodies were determined with flow cytometry using K562 cells that have been transfected with a CD11b expression vector. As shown in FIG. 10, all humanized anti-CD11b antibodies tested were able to bind the CD11b expressing K562 cells. In contrast, these antibodies did not bind un-transfected K562 cells. These results show that humanized anti-CD11b antibodies can specifically bind the CD11b epitope. It should be noted that all combination or permutations of the heavy chains and light chains bind tightly to CD11b. Similarly, these humanized antibodies also bind specifically to CD11b on HepG2 cells.

Embodiments of the invention may use any of the above anti-CD11b antibodies, or an antigen-binding portion thereof, that comprises at least one of a heavy-chain complementarity determining region 1 (HCDR1) consisting of the amino acid residues of NYWIN (SEQ ID NO:1) or GFSLTSNSIS (SEQ ID NO:2); a heavy chain CDR2 (HCDR2) consisting of the amino acid residues of NIYPSDTYINHNQKFKD (SEQ ID NO:3) or AIWSGGGTDYNSDLKS (SEQ ID NO:4); and a heavy chain CDR3 (HCDR3) consisting of the amino acid residues of SAYANYFDY (SEQ ID NO:5) or RGGYPYYFDY (SEQ ID NO:6); and at least one of a light chain CDR1 (LCDR1) consisting of the amino acid residues of RASQNIGTSIH (SEQ ID NO:7) or KSSQSLLYSENQENYLA (SEQ ID NO:8); a light chain CDR2 (LCDR2) consisting of the amino acid residues of YASESIS (SEQ ID NO:9) or WASTRQS (SEQ ID NO:10); and a light chain CDR3 (LCDR3) consisting of the amino acid residues QQSDSWPTLT (SEQ ID NO:11) or QQYYDTPLT (SEQ ID NO:12).

In some embodiments of the present invention, an anti-CD11b antibody or an antigen-binding portion thereof, comprises (i) a heavy chain variable region comprising a heavy chain variable region comprising H-CDR1 comprising SEQ ID NO:1, H-CDR2 comprising SEQ ID NO:3 and H-CDR3 comprising SEQ ID NO:5, and (ii) light chain variable regions comprising L-CDR1 comprising SEQ ID NO:7, L-CDR2 comprising SEQ ID NO:9 and L-CDR3 comprising SEQ ID NO:11; or (iii) a heavy chain variable region comprising a heavy chain variable region comprising H-CDR1 comprising SEQ ID NO:2, H-CDR2 comprising SEQ ID NO:4 and H-CDR3 comprising SEQ ID NO:6, and (iv) light chain variable regions comprising L-CDR1 comprising SEQ ID NO:8, L-CDR2 comprising SEQ ID NO:10 and L-CDR3 comprising SEQ ID NO:12.

In some embodiments of the present invention, a humanized anti-CD11b antibody or an antigen-binding portion thereof, comprises:
(a) a heavy chain variable region comprising an amino acid sequence consisting of SEQ ID NO:13, and a light chain variable region comprising an amino acid sequence consisting of SEQ ID NO:23;
(b) a heavy chain variable region comprising an amino acid sequence consisting of SEQ ID NO:14, and a light chain variable region comprising an amino acid sequence consisting of SEQ ID NO:24;
(c) a heavy chain variable region comprising an amino acid sequence consisting of SEQ ID NO:15, and a light chain variable region comprising an amino acid sequence consisting of SEQ ID NO:25;
(d) a heavy chain variable region comprising an amino acid sequence consisting of SEQ ID NO:16, and a light chain variable region comprising an amino acid sequence consisting of SEQ ID NO:26;
(e) a heavy chain variable region comprising an amino acid sequence consisting of SEQ ID NO:17, and a light chain variable region comprising an amino acid sequence consisting of SEQ ID NO:27;
(f) a heavy chain variable region comprising an amino acid sequence consisting of SEQ ID NO:18, and a light chain variable region comprising an amino acid sequence consisting of SEQ ID NO:28;
(g) a heavy chain variable region comprising an amino acid sequence consisting of SEQ ID NO:19, and a light chain variable region comprising an amino acid sequence consisting of SEQ ID NO:29;
(h) a heavy chain variable region comprising an amino acid sequence consisting of SEQ ID NO:20, and a light chain variable region comprising an amino acid sequence consisting of SEQ ID NO:30;
(i) a heavy chain variable region comprising an amino acid sequence consisting of SEQ ID NO:21, and a light chain variable region comprising an amino acid sequence consisting of SEQ ID NO:31; or
(j) a heavy chain variable region comprising an amino acid sequence consisting of SEQ ID NO:22, and a light chain variable region comprising an amino acid sequence consisting of SEQ ID NO:32.

### Treatment with Anti-CD11b Antibody Enhanced Antigen-Presenting Capacity of CD11b+ Immune Cells

To evaluate the therapeutic effects of anti-CD11b antibodies against chronic HBV infection, we utilized an HBV-carrier mouse model developed by hydrodynamic injection (HDI) of the pAAV/HBV1.2 plasmid into CBA/caJ mice. Briefly, ten micrograms of pAAV/HBV1.2 DNA was injected hydrodynamically into the tail veins of male CBA/caJ mice. After injection, the mice were regularly bled to monitor the serum levels of HBsAg and HBV DNA. (Huang et al., Proc. Natl. Acad. Sci. U.S.A. 2006 Nov. 21; 103(47):17862-17867).

The HBV carrier mice expressed hepatitis B surface antigen (HBsAg), hepatitis B e antigen (HBeAg), hepatitis B core antigen (HBcAg), and high levels of serum HBV DNA, but with normal levels of serum alanine aminotransferase (ALT) and without significant inflammation in the liver. The characteristics of this mouse model for HBV persistence are analogous to those of human chronic HBV infections in the immune tolerant stage. (Chou et al., Proc Natl Acad Sci USA. 2015 Feb. 17; 112(7):2175-80).

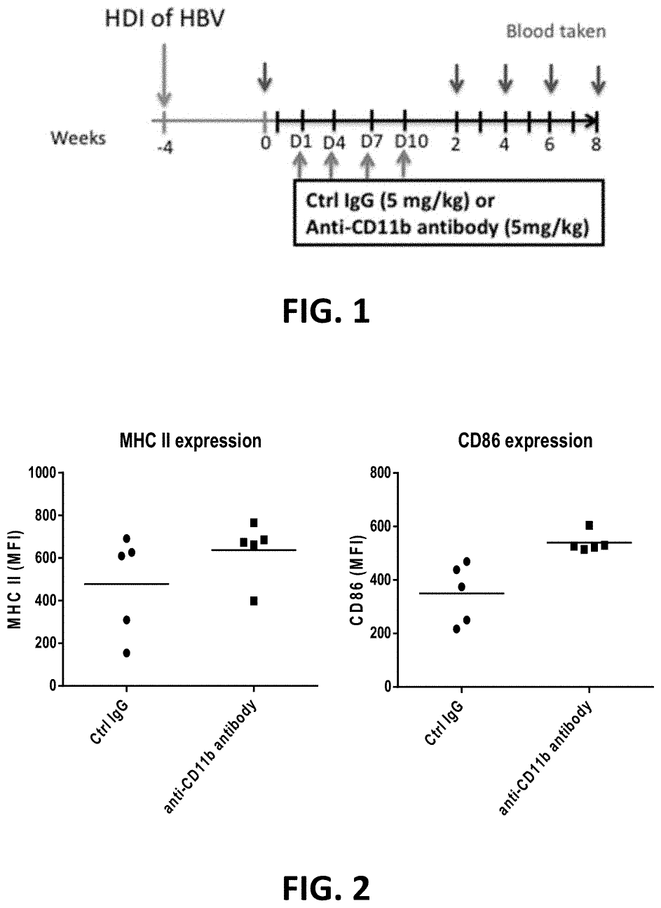

As shown in FIG. 1, HBV carrier mice (4 weeks after hydrodynamic injection) were divided into two groups and treated with 5 mg/kg of a control IgG (ctrl IgG) or an anti-CD11b antibody. Injections were repeated every 3-4 days for 4 times. Blood samples were collected for analyses at weeks 2, 4, 6, and 8.

The activation status of CD11b+ peripheral blood mononuclear cells (PBMC) in HBV carrier mice was evaluated at two weeks after the initial antibody treatments. Compared to the Ctrl IgG-treated mice, administration of anti-CD11b antibody resulted in an increase in the expression levels of MHC II and CD86 in CD11b+ PBMCs (FIG. 2). These results indicate that treatments with anti-CD11b antibodies can enhance antigen-presenting capacity of CD11b+ immune cells, which will be favorable for innate and adoptive immune activation to eliminate virus in the HBV carrier mice. Therefore, anti-CD11b antibodies may be useful therapeutics for treating HBV infections.

### Anti-CD11b Antibody Treatment Leads to Accelerated Clearance of HBV Infection

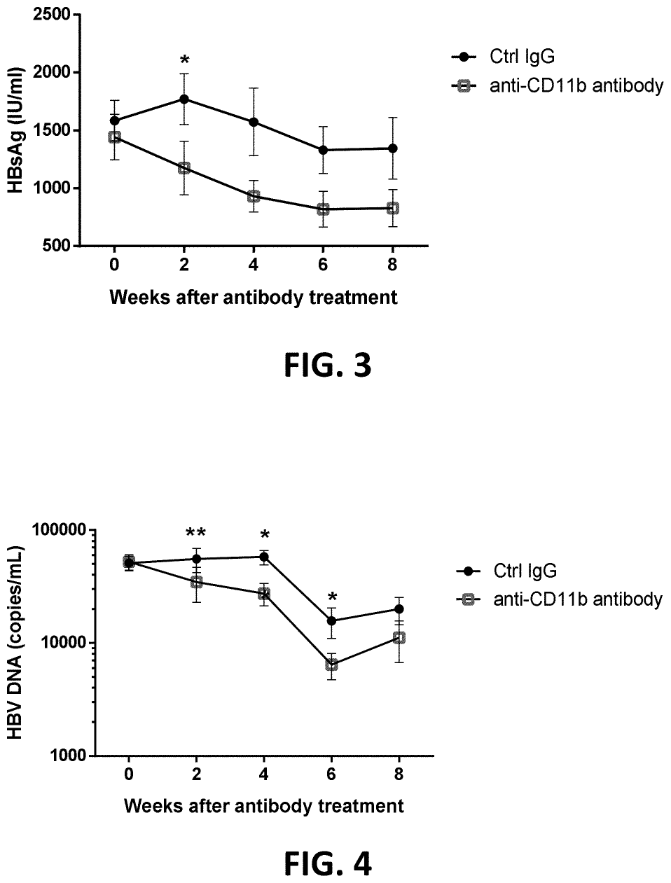

The therapeutic effects of anti-CD11b antibodies against chronic HBV infection in HBV carrier mice were examined. Treatment with anti-CD11b antibody significantly inhibited serum HBsAg levels two weeks after antibody injection (FIG. 3), as compared with the ctrl IgG treatment group. Anti-CD11b antibody treatment also dramatically reduced the levels of HBV replication (evidenced by lower DNA levels) two weeks after the initial antibody injection (FIG. 4). Sustained viral suppression was observed in mice that received anti-CD11b antibody for several weeks. In addition, serum HBsAg and HBV DNA rebound did not occur in most mice treated with anti-CD11b antibody (FIGS. 3 and 4). No rebound of the infection indicates that the viruses are eliminated by the enhanced immune response, rather than temporarily suppressed. These results show that anti-CD11b antibody treatments can induce accelerated clearance of HBV and return of the infection does not occur.

### Enhanced Antigen-Presenting Capacity by Anti-CD11b Antibody Treatment is Associated with Clearance of HBV Infection

As noted above, treatments with anti-CD11b antibodies can enhance antigen-presenting capacity of CD11b+ immune cells, leading to enhanced immune responses. To investigate whether there is a relationship between the clearance of HBV infection and antigen-presenting capacity of CD11b+ immune cells, the correlation between serum HBV DNA, MHC II and CD86 expression in CD11b+ PBMCs were assessed.

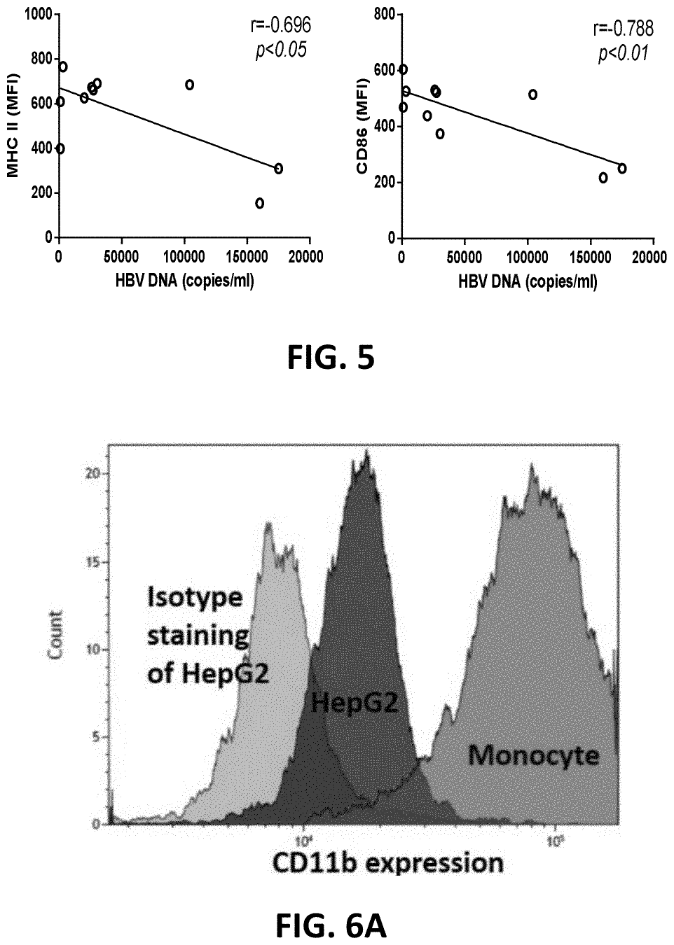

As shown in FIG. 5, increased surface expression of MHCII and CD86 in the CD11b+ PBMCs is negatively correlated with levels of the serum HBV DNA. These results indicate that enhanced antigen-presenting capacity by anti-CD11b antibody treatment is associated with enhanced clearance of HBV infection.

### Anti-CD11b Antibodies Inhibit the HBsAg Production of HBV-Transfected Human Hepatoma HepG2 Cell Line and Induce DNA Deaminases Including Apolipoprotein B mRNA Editing Enzyme, Catalytic Polypeptide-Like (APOBEC) Proteins that May Degrade HBV Covalently Closed Circular DNA (cccDNA)

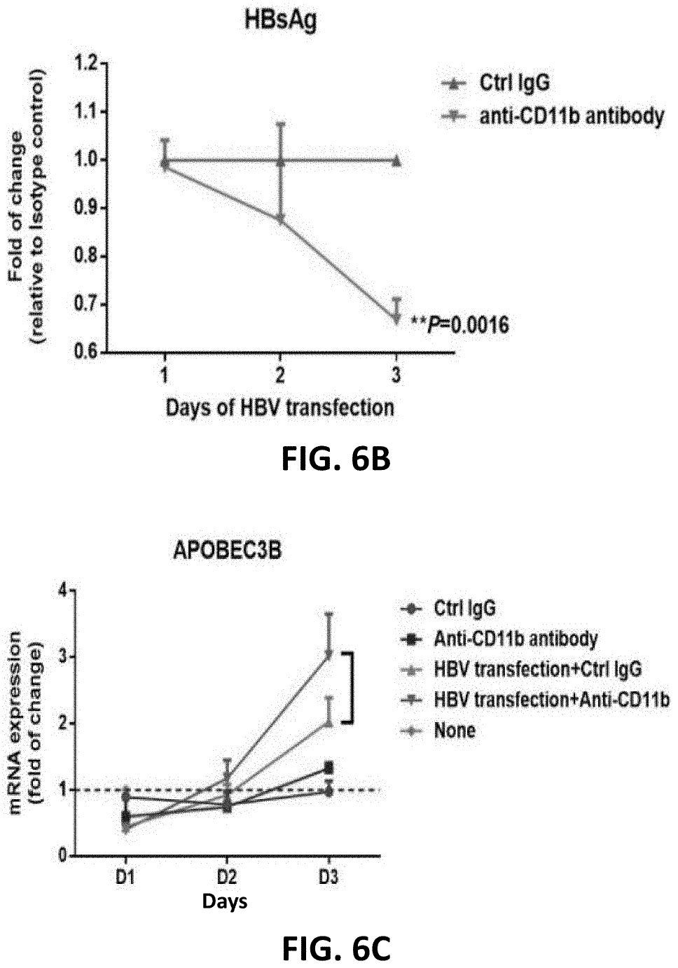

In addition to the above HBV mouse model, the efficacies of anti-CD11b antibodies in the treatment of HBV infection were also investigated using human HBV infected HepG2 cells. The CD11b expression on cell surface was evaluated by flow cytometry. As shown in FIG. 6A, the expression of CD11b on HepG2 cells is much lower than that on human monocytes. Human HepG2 cells were transfected with HBV plasmids, and the titers of HBsAg in culture soup were evaluated with HBsAg quantitative ELISA kit. After 3-day anti-CD11b antibody treatment, the titers of HBsAg of HBV-transfected HepG2 cells were rapidly and significantly decreased (FIG. 6B).

APOBEC3B is a cytidine deaminase that has been found to be a cellular restriction factor for HBV because APOBEC3B can edit HBV cccDNA in the nucleus, leading to its degradation. (Chen et al., Antiviral Res., 2018 January; 149:16-25). The RNA of APOBEC3B expression was increased in the anti-CD11b antibodies-treated HBV-transfected HepG2 cells (FIG. 6C). These results suggest that a non-cytolytic mechanism is at least partially responsible for the clearance of HBsAg after treatment with anti-CD11b antibodies. In addition, treatment with anti-CD11b antibodies may involve functional inhibition and/or degradation of HBV cccDNA, which may be targeted by anti-CD11b antibodies through epigenetic modifications, induction of DNA deaminases APOBEC proteins, microRNAs, inhibition of conversion from relaxed circular DNA (rcDNA) to cccDNA, blocking the rcDNA transportation into nucleus, and/or inhibition of cccDNA transcription.

### Treatment with Anti-CD11b Antibody Induce HBV DNA Reduction in the Liver

The above results indicate that anti-CD11b antibodies can significantly reduce the levels of HBsAg and DNA. Whether this is due to temporary suppression of HBV (e.g., rendering the viruses dormant) or long-term effects (e.g., reduction or elimination of HBV from liver) is further investigated by assessing the levels of HBV DNA in the liver long after the treatment. For example, 36 weeks after anti-CD11b antibody treatment, resident HBV DNA in liver was quantified. Briefly, liver was ground in liquid nitrogen and the total liver genomic DNA (gDNA) was extracted. HBV DNA was detected with real time PCR using HBx specific primers (Forward primer: 5′-CCGATCCATACTGCGGAAC-3′, SEQ ID NO: 33; Reverse primer: 5′-GCAGAGGTGAAGCGAAGTGCA-3′, SEQ ID NO: 34).

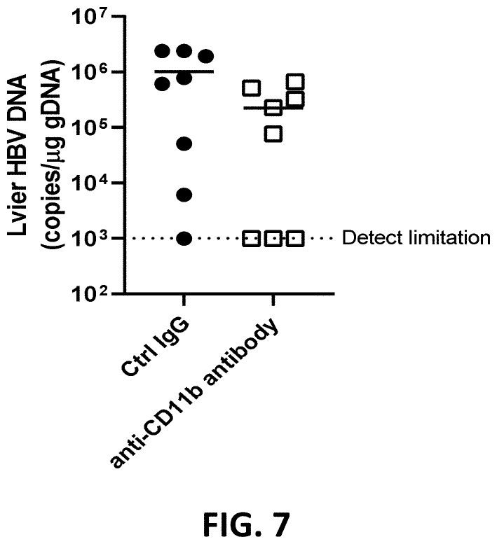

FIG. 7 shows the results from this study. The HBV DNA was represented as numbers of copies in 1 μg of mice gDNA. The mean value of HBV DNA was 1.01×10⁶ and 2.26×10⁵ in Ctrl IgG and anti-CD11b antibody treated groups, respectively. Thus, the copy numbers of HBV in the anti-CD11b antibody treated group is significantly lower (about 22%) than that of the control IgG treated group. The liver HBV clearance rate was 12.5% (one in eight mice HBV DNA was undetectable) and 37.5% (three in eight mice HBV DNA was undetectable) in Ctrl IgG and anti-CD11b antibody treated groups, respectively. These results indicate that the liver HBV DNA was significantly reduced in mice treated with anti-CD11b antibody. More importantly, these results are at a long time after the treatment, suggesting that the treatment effects are durable and are due to clearance of the viruses from liver, rather than due to temporary suppression of the viruses. Therefore, methods of the invention using anti-CD11b antibodies are very promising for the treatment of HBV infections.

### Materials and Methods

### Hydrodynamic Injection-Based HBV Carrier Mice and Treatment Protocol

A total of 10 μg of pAAV/HBV1.2 dissolved in 8% body weight of PBS was injected into the tail vein of 6- to 8-week-old CBA/caJ mice. The total volume was delivered within 5-7 seconds. (Chou et al., Proc Natl Acad Sci U.S.A., 2015; 112(7): 2175-80). pAAV/HBV1.2 contains an HBV fragment spanning nucleotides 1400-3182/1-1987 flanked by inverted terminal repeats of AAV. (Huang et al., Proc Natl Acad Sci U.S.A., 2006, 103(47): 17862-17867). Four weeks later, mice were intraperitoneally (i.p.) treated with an 5 mg/kg of anti-CD11b Ab or isotype control Ab. Injections were repeated every 3-4 days for 4 times. All mice were maintained under specific pathogen-free conditions in the National Taiwan University College of Medicine Laboratory of Animal Center. The experiments were conducted in accordance with the guidelines for experimental animal use specified by the National Taiwan University College of Medicine.

### Serum HBsAg and HBV DNA Analysis

Serum hepatitis B surface antigen (HBsAg) was quantitated using an AXSYM® system kit (Abbott Diagnostika, Abbot Park, Ill., USA). Assays were performed according to the manufacture's protocols. To detect serum HBV DNA, total DNA was extracted from each serum sample and HBV DNA was detected by a real-time PCR with HBx specific primers.

### Liver HBV DNA Analysis

To detect liver HBV DNA, liver was ground in liquid nitrogen and the total liver genomic DNA (gDNA) was extracted using a commercially available kit. HBV DNA was detected with real time PCR using HBx specific primers (described above).

### Flow Cytometry Analysis

The antigen-presenting capacity of CD11b+ PBMCs was examined for the expression of MHC II and CD86 markers. PBMCs were incubated with fluorescently-conjugated anti-CD11b (M1/70, ICRF44), CD86 (GL-1), MHC II (M5/114.15.2) or an appropriate isotype control antibody for 20 min. Samples were run on a Beckman Coulter (Indianapolis, Ind., USA) CytoFLEX flow cytometer, and data acquisition and analysis were performed using Kaluza analysis software version 2.0 from Beckman Coulter.

### HepG2 Cell Infection Assay

HepG2 cells were maintained with 10% DMEM medium and transfected with pAAV/HBV1.2 plasmid (provided by Dr. PEI-JER CHEN, National Taiwan University, Taipei, Taiwan) using Lipofectamine3000 for 8-hr incubation. After transfection, cells were rinsed with PBS three time and were continually cultured with 10% DMEM medium with/without anti-human CD11b antibodies (10 μg/ml). The cell culture soup was collected daily and the titer of HBsAg were measured by HBsAg quantitative ELISA kit, Rapid-II (Beacle Inc., Kyoto, Japan). The RNA of HepG2 cells were extracted by RNeasy Mini Kit and treated with DNase to remove genomic DNA contamination. The gene expressions of APOBEC3 were evaluated by real-PCR as previously described (J. Lucifora et al., Specific and nonhepatotoxic degradation of nuclear hepatitis B virus, Science. 2014 Mar. 14; 343(6176):1221-8).

### Statistical Analysis

Data were analyzed using Prism 6.0 (GraphPad) and expressed as the mean±SEM. Comparisons between groups were performed using the Student t test. Correlations were determined using the Pearson's correlation coefficient. A p value<0.05 was considered significant.

1. A method for treating hepatitis B virus (HBV) infection, comprising: administering to a subject in need thereof a pharmaceutical composition comprising an effective amount of an antibody against CD11b or a binding fragment thereof.

2. The method according to claim 1, wherein the antibody against CD11b is a monoclonal antibody.

3. The method according to claim 1, wherein the antibody against CD11b comprises a heavy-chain complementarity determining region 1 (HCDR1) consisting of the amino acid residues of NYWIN (SEQ ID NO:1) or GFSLTSNSIS (SEQ ID NO:2); a heavy chain CDR2 (HCDR2) consisting of the amino acid residues of NIYPSDTYINHNQKFKD (SEQ ID NO:3) or AIWSGGGTDYNSDLKS (SEQ ID NO:4); and a heavy chain CDR3 (HCDR3) consisting of the amino acid residues of SAYANYFDY (SEQ ID NO:5) or RGGYPYYFDY (SEQ ID NO:6); and a light chain CDR1 (LCDR1) consisting of the amino acid residues of RASQNIGTSIH (SEQ ID NO:7) or KSSQSLLYSENQENYLA (SEQ ID NO:8); a light chain CDR2 (LCDR2) consisting of the amino acid residues of YASESIS (SEQ ID NO:9) or WASTRQS (SEQ ID NO:10); and a light chain CDR3 (LCDR3) consisting of the amino acid residues QQSDSWPTLT (SEQ ID NO:11) or QQYYDTPLT (SEQ ID NO:12).

4. The method according to claim 1, wherein the antibody against CD11b comprises:
(a) a heavy chain variable region comprising the sequence of SEQ ID NO:13, and a light chain variable region comprising the sequence of SEQ ID NO:23;
(b) a heavy chain variable region comprising the sequence of SEQ ID NO:14, and a light chain variable region comprising the sequence of SEQ ID NO:24;
(c) a heavy chain variable region comprising the sequence of SEQ ID NO:15, and a light chain variable region comprising the sequence of SEQ ID NO:25;
(d) a heavy chain variable region comprising the sequence of SEQ ID NO:16, and a light chain variable region comprising the sequence of SEQ ID NO:26;
(e) a heavy chain variable region comprising the sequence of SEQ ID NO:17, and a light chain variable region comprising the sequence of SEQ ID NO:27;
(f) a heavy chain variable region comprising the sequence of SEQ ID NO:18, and a light chain variable region comprising the sequence of SEQ ID NO:28;
(g) a heavy chain variable region comprising the sequence of SEQ ID NO:19, and a light chain variable region comprising the sequence of SEQ ID NO:29;
(h) a heavy chain variable region comprising the sequence of SEQ ID NO:20, and a light chain variable region comprising the sequence of SEQ ID NO:30;
(i) a heavy chain variable region comprising the sequence of SEQ ID NO:21, and a light chain variable region comprising the sequence of SEQ ID NO:31; or
(j) a heavy chain variable region comprising the sequence of SEQ ID NO:22, and a light chain variable region comprising the sequence of SEQ ID NO:32.

5. (canceled)


SEQUENCE LISTING (34 sequences)

SEQ ID NO: 1 - protein, 5 aa, Artificial
  H-CDR1
NYWIN

SEQ ID NO: 2 - protein, 10 aa, artificial
  H-CDR1
GFSLTSNSIS

SEQ ID NO: 3 - protein, 17 aa, artificial
  H-CDR2
NIYPSDTYINHNQKFKD

SEQ ID NO: 4 - protein, 16 aa, artificial
  H-CDR2
AIWSGGGTDYNSDLKS

SEQ ID NO: 5 - protein, 9 aa, artificial
  H-CDR3
SAYANYFDY

SEQ ID NO: 6 - protein, 10 aa, artificial
  H-CDR3
RGGYPYYFDY

SEQ ID NO: 7 - protein, 11 aa, artificial
  L-CDR1
RASQNIGTSIH

SEQ ID NO: 8 - protein, 17 aa, artificial
  L-CDR1
KSSQSLLYSENQENYLA

SEQ ID NO: 9 - protein, 7 aa, artificial
  L-CDR2
YASESIS

SEQ ID NO: 10 - protein, 7 aa, artificial
  L-CDR2
WASTRQS

SEQ ID NO: 11 - protein, 10 aa, artificial
  L-CDR3
QQSDSWPTLT

SEQ ID NO: 12 - protein, 9 aa, artificial
  L-CDR3
QQYYDTPLT

SEQ ID NO: 13 - protein, 118 aa, artificial
  Heavy chain variable region of the humanized anti-CD11b antibodies-VH1
QVQLVQSGAEVKKPGASVKVSCKASGYTFTNYWINWVRQAPGQGLEWMGNIYPSDTYINHNQKFKDRVTMTRDTSTSTVYMELSSLRSEDTAVYYCARSAYANYFDYWGQGTLVTVSS

SEQ ID NO: 14 - protein, 118 aa, artificial
  Heavy chain variable region of the humanized anti-CD11b antibodies-VH2
QVQLVQSGAEVKKPGSSVKVSCKASGGTFSNYWINWVRQAPGQGLEWMGNIYPSDTYINHNQKFKDRVTITADKSTSTAYMELSSLRSEDTAVYYCATSAYANYFDYWGQGTLVTVSS

SEQ ID NO: 15 - protein, 118 aa, artificial
  Heavy chain variable region of the humanized anti-CD11b antibodies-VH3
QVQLVQSGAEVKKPGASVKVSCKASGYTFTNYWINWVRQATGQGLEWMGNIYPSDTYINHNQKFKDRVTMTRNTSISTAYMELSSLRSEDTAVYYCARSAYANYFDYWGQGTLVTVSS

SEQ ID NO: 16 - protein, 118 aa, artificial
  Heavy chain variable region of the humanized anti-CD11b antibodies-VH4
QVQLVQSGAEVKKPGASVKVSCKASGYTFTNYWINWVRQAPGQRLEWMGNIYPSDTYINHNQKFKDRVTITRDTSASTAYMELSSLRSEDTAVYYCARSAYANYFDYWGQGTLVTVSS

SEQ ID NO: 17 - protein, 118 aa, artificial
  Heavy chain variable region of the humanized anti-CD11b antibodies-VH5
QVQLVQSGAEVKKPGATVKISCKVSGYTFTNYWINWVQQAPGKGLEWMGNIYPSDTYINHNQKFKDRVTITADTSTDTAYMELSSLRSEDTAVYYCARSAYANYFDYWGQGTLVTVSR

SEQ ID NO: 18 - protein, 117 aa, artificial
  Heavy chain variable region of the humanized anti-CD11b antibodies-HC1
QVQLQESGPGLVKPSETLSLTCTVSGFSLTSNSISWIRQPPGKGLEWIGAIWSGGGTDYNSDLKSRVTISVDTSKNQFSLKLSSVTAADTAVYYCARGGYPYYFDYWGQGTLVTVSS

SEQ ID NO: 19 - protein, 117 aa, artificial
  Heavy chain variable region of the humanized anti-CD11b antibodies-HC2
QVQLQESGPGLVKPSGTLSLTCAVYGFSLTSNSISWIRQPPGKGLEWIGAIWSGGGTDYNSDLKSRVTISVDTSKNQFSLKLSSVTAADTAVYYCARGGYPYYFDYWGQGTMVTVSS

SEQ ID NO: 20 - protein, 117 aa, artificial
  Heavy chain variable region of the humanized anti-CD11b antibodies-HC3
QVQLQQWGAGLLKPSETLSLTCAVYGFSLTSNSISWIRQPPGKGLEWIGAIWSGGGTDYNSDLKSRVTISVDTSKNQFSLKLSSVTAADTAVYYCARGGYPYYFDYWGQGTLVTVSS

SEQ ID NO: 21 - protein, 117 aa, artificial
  Heavy chain variable region of the humanized anti-CD11b antibodies-HC4
EVQLVESGGGLVQPGGSLRLSCAASGFSLTSNSISWVRQAPGKGLEWVSAIWSGGGTDYNSDLKSRFTISRDNSKNTLYLQMNSLRAEDTAVYYCARGGYPYYFDYWGQGTLVTVSS

SEQ ID NO: 22 - protein, 117 aa, artificial
  Heavy chain variable region of the humanized anti-CD11b antibodies-HC5
EVQLVETGGGLIQPGGSLRLSCAASGFSLTSNSISWVRQAPGKGLEWVSAIWSGGGTDYNSDLKSRFTISRDNSKNTLYLQMNSLRAEDTAVYYCARGGYPYYFDYWGQGTLVTVSS

SEQ ID NO: 23 - protein, 108 aa, artificial
  Light chain variable region of the humanized anti-CD11b antibodies-VL1
EIVLTQSPDFQSVTPKEKVTITCRASQNIGTSIHWYQQKPDQSPKLLIKYASESISGVPSRFSGSGSGTDFTLTINSLEAEDAATYYCQQSDSWPTLTFGQGTKVEIK

SEQ ID NO: 24 - protein, 108 aa, artificial
  Light chain variable region of the humanized anti-CD11b antibodies-VL2
EIVMTQSPATLSVSPGERATLSCRASQNIGTSIHWYQQKPGQAPRLLIYYASESISGIPARFSGSGSGTEFTLTISSLQSEDFAVYYCQQSDSWPTLTFGQGTKLEIK

SEQ ID NO: 25 - protein, 108 aa, artificial
  Light chain variable region of the humanized anti-CD11b antibodies-VL3
DIQMTQSPSSLSASVGDRVTITCRASQNIGTSIHWYQQKPGKAPKLLIYYASESISGVPSRFSGSGSGTDFTLTISSLQPEDFATYYCQQSDSWPTLTFGGGTKVEIK

SEQ ID NO: 26 - protein, 108 aa, artificial
  Light chain variable region of the humanized anti-CD11b antibodies-VL4
EIVLTQSPATLSLSPGERATLSCRASQNIGTSIHWYQQKPGQAPRLLIYYASESISGIPARFSGSGSGTDFTLTISSLEPEDFAVYYCQQSDSWPTLTFGGGTKVEIK

SEQ ID NO: 27 - protein, 108 aa, artificial
  Light chain variable region of the humanized anti-CD11b antibodies-VL5
EIVLTQSPGTLSLSPGERATLSCRASQNIGTSIHWYQQKPGQAPRLLIYYASESISGIPDRFSGSGSGTDFTLTISRLEPEDFAVYYCQQSDSWPTLTFGQGTKLEIK

SEQ ID NO: 28 - protein, 113 aa, artificial
  Light chain variable region of the humanized anti-CD11b antibodies-LC1
DIVMTQSPDSLAVSLGERATINCKSSQSLLYSENQENYLAWYQQKPGQPPKLLIYWASTRQSGVPDRFSGSGSGTDFTLTISSLQAEDVAVYYCQQYYDTPLTFGQGTKVEIK

SEQ ID NO: 29 - protein, 113 aa, artificial
  Light chain variable region of the humanized anti-CD11b antibodies-LC2
DIVMTQSPLSLPVTPGEPASISCKSSQSLLYSENQENYLAWYLQKPGQSPQLLIYWASTRQSGVPDRFSGSGSGTDFTLKISRVEAEDVGVYYCQQYYDTPLTFGGGTKVEIK

SEQ ID NO: 30 - protein, 113 aa, artificial
  Light chain variable region of the humanized anti-CD11b antibodies-LC3
DIVMTQSPLSLSVTPGQPASISCKSSQSLLYSENQENYLAWYLQKPGQSPQLLIYWASTRQSGVPDRFSGSGSGTDFTLKISRVEAEDVGVYYCQQYYDTPLTFGQGTKVEIK

SEQ ID NO: 31 - protein, 113 aa, artificial
  Light chain variable region of the humanized anti-CD11b antibodies-LC4
DVVMTQSPLSLPVTLGQPASISCKSSQSLLYSENQENYLAWFQQRPGQSPRRLIYWASTRQSGVPDRFSGSGSGTDFTLKISRVEAEDVGVYYCQQYYDTPLTFGQGTKLEIK

SEQ ID NO: 32 - protein, 113 aa, artificial
  Light chain variable region of the humanized anti-CD11b antibodies-LC5
DIVMTQTPLSSPVTLGQPASISCKSSQSLLYSENQENYLAWLQQRPGQPPRLLIYWASTRQSGVPDRFSGSGAGTDFTLKISRVEAEDVGVYYCQQYYDTPLTFGQGTKLEIK

SEQ ID NO: 33 - DNA, 19 bp, artificial
  HBx forward primer
ccgatccatactgcggaac

SEQ ID NO: 34 - DNA, 21 bp, artificial
  HBx reverse primer
gcagaggtgaagcgaagtgca